(19) United States
(12) Patent Application Publication
(10) Pub. No.: US 2023/0070870 A1
(43) Pub. Date: Mar. 9, 2023

(54) Covid Nasal Spray

(71) Applicant: Robert V. West, III, San Antonio, TX (US)

(72) Inventor: Robert V. West, III, San Antonio, TX (US)

(21) Appl. No.: 17/300,640

(22) Filed: Sep. 9, 2021

(51) Int. Cl. A61K 31/14; A61K 9/00

(52) U.S. Cl. CPC A61K 31/14; A61K 9/0073

(57) ABSTRACT

Direct instillation of a Chlorinated Solution into the Nasal passages to inactivate Covid 19 by way of Nasal spray of a Sodium Hypochlorite solution. For topical elimination of Covid 19, Solutions of 5-12.5 Parts Per Million; 5-12.5 mg. per Liter; 5-12.5% Solutions; are generally effective and considered safe by the EPA and FDA for indirect human consumption. While stronger solutions are recognized for eradication of the Coronavirus on inorganic impervious surfaces, these more dilute solutions have not previously been used for this application to the Nasal Mucosa and Nasopharynx of Humans. This new and revolutionary Process for use of Sodium Hypochlorite offers an alternative therapeutic and prophylactic modality in the war against Coronavirus (Covid-19). Sodium Hypochlorite is well known for disinfecting hard surfaces and its antimicrobial effects in higher concentrations. However, currently there are no biological products to suppress and eradicate Covid-19 in humans, besides Vaccination, Social Distancing, and Face Masks.

### BACKGROUND OF THE INVENTION

The World has been immersed in a pandemic from a new Coronavirus since the winter months of 2019. The origin of the pathogen was China. The Pathogen was classified as Novel because it contains unique biologic markers and properties when compared with previous recognized Coronaviruses. The unique property enabling Worldwide spread of this Virus is the presence of the M-Spike protein on the Virus particle. The M-Spike protein which facilitates entry into host cells, by penetrating the cell wall of susceptible tissues. The name for this new entity is Covid 19, “C-19”.

The current Novel Process attacks the Virus on nasal and nasopharyngeal pharyngeal mucosal surfaces before the unique Spike protein locks it onto a target cell receptor. After docking, the C-19 virus penetrates the nerve cells of the nasal mucosa and nerve endings of the Olfactory Nerve and inserts it messenger RNA. The most critical target for the eradication and suppression of primary Infections or Vaccine “break thru” infection are the intra-nasal Olfactory Nerve origins on the surface of the Nasal passages and the Nasopharynx. The dendritic terminals of this Nerve originate and are concentrated on the nasal mucosa. Once the Covid-19 Virus Spike attaches and enters the Olfactory Nerve cell, it travels centrally along the nerve axon into the Central Nervous System causing encephalitis (headaches) and loss of Olfactory function (scent). The Olfactory Nerve is also known as Cranial Nerve II. The Covid-19 virus can spread from there systemically or reside there dormant in the Olfactory Nerve as a facultative intracellular pathogen. In this location as a passenger, it is prone to periodic flares and spontaneous outbreaks. Covid Nasal Spray acts directly within the Nose aided by the Body's immune system to attack and kill the Virus in the nostrils either before entry or release from Nasal tissues. While residing inside the Nerve Cells, the virus is protected from attack by the Nerve cellular membrane and the Blood Brain Barrier. Covid-19 clinical infection appears as a Flu-Like Illness during a symptomatic infection when shed onto the mucosa surfaces of Nasal passages and the Nasopharynx. During this phase it can be detected in the Nose on the PCR Antigen Test. The shedding occurs during a primary infection or during an asymptomatic “shedding” phase.

Despite the availability of numerous vaccines, this cycle persists in 2021 with no know direct way to interrupt it except when the C-19 virus is exposed on mucosal surfaces or in the blood stream. At the time of this writing, numerous new strains of Covid 19 are emerging worldwide. New mutations are suggested every 4-5 months. Physical barriers such as masks and Vaccines are partially effective, but until now there are no topical modalities with direct Virucidal properties such as the present Process.

Aerosolization refers to a process of intentionally suspending particles and transmitting a solution into a moving stream of air for the purpose of delivering the particles or composition to a given location. In this context, positive pressure is required to project a spray of the solution into the Nasal passages and towards the Nasopharynx. A handheld squeeze bottle is most efficient and convenient delivery system.

### SUMMARY OF THE INVENTION

Despite the initial optimism that the advent of Vaccines would eliminate or curtail future cases of Covid-19, many patients and the Scientific community are aware of many “break thru” cases and new variants. Vaccine success as to eradication against Delta, Omicron, and other variants is deemed to be about 50%. Recurrences of the Covid variants in vaccinated persons are sometimes asymptomatic or low grade, however the contagion remains real and pervasive, presenting a serious public health threat. Dr. West's Process is a safe, convenient, and easy to use nasal spray solution to interrupt the disease process in infected or exposed persons. While face masks may protect entry of the Virus into the Nasal passages, this Novel Process creates a second stage aimed at eradicating the Virus before it invades the tissue of the Nose and Nasopharynx. Just as the virus persists on hard surfaces for up 72 hours, the Virus exists on the human mucosal membranes before infection. Despite social distancing and the wearing of masks, this Process is necessary to interrupt the life cycle of this Covid-19 pathogen. After transmission or shedding, the Covid-19 virus resides on the mucous membranes of the Nose and Nasopharynx and is subject to the viricidal properties of this product, Covid Nasal Spray.

The inventor Robert V. West III, M.D. is a Texas Physician and Board Certified in Emergency Physician practicing in the State of Texas for 42 years. Currently, due to recurring waves of Coronavirus strains and the advent of Telemedicine, nine states have extended their State Medical Licenses to Dr. West. He treats patients Nationwide and treats their C-19 infections as well. Dr. West has a B.A. in Natural Sciences and Doctor of Medicine, both from the University of Texas.

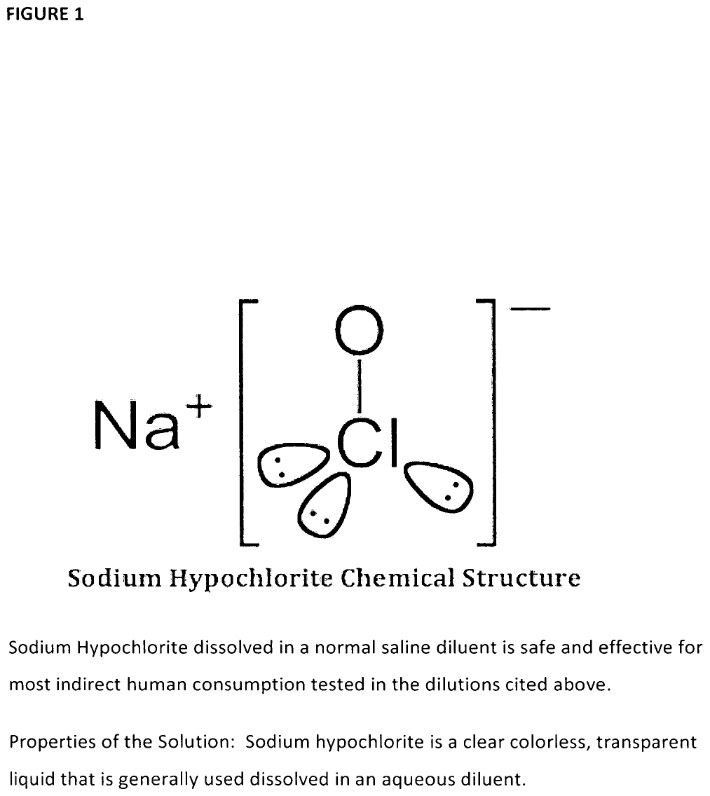

1. This Invention relates to the process and delivery of a dilute Sodium Hypochlorite solution ranging from 5 P.P.M. to 12.5 P.P.M.; 5-12 mg. per liter; 5-12.5 mg. % into the human Nasal Passages and Nasopharynx using an aerosol spray bottle or inhaler. (See FIG. 1))

2. A Novel, Non-Obvious process to inactivate, retard, and suppress Covid-19 replication in nasal tissues reducing infection, transmission and recurrence of the Coronavirus, by the topical delivery of a dilute Sodium Hypochlorite solution to the mucosa of the human nasopharynx
